(19) United States
(12) Patent Application Publication
(10) Pub. No.: US 2025/0296902 A1
(43) Pub. Date: Sep. 25, 2025

(54) SYNTHETIC METHANOL HAVING LOW DEUTERIUM CONTENT FROM NON-FOSSIL RESOURCES

(71) Applicant: BASF SE, Ludwigshafen am Rhein (DE)

(72) Inventors: Thomas WEISS, Ludwigshafen am Rhein (DE); Stephan HUEFFER, Ludwigshafen am Rhein (DE)

(21) Appl. No.: 18/862,184

(22) PCT Filed: Apr. 24, 2023
(86) PCT No.: PCT/EP2023/060570

§ 371 Date: Nov. 1, 2024

(30) Foreign Application Priority Data
May 2, 2022 (EP) 22171092.4

(51) Int. Cl. C07C 29/151; C07C 29/38; C07C 41/09; C07C 45/27; C07C 51/16; C07C 67/08; C07C 209/16

(52) U.S. Cl. CPC C07C 29/1518; C07C 29/38; C07C 41/09; C07C 45/27; C07C 51/16; C07C 67/08; C07C 209/16

(57) ABSTRACT

A Process for making methanol having a deuterium content below 90 ppm, based on the total hydrogen content, comprising the steps: (a) providing hydrogen with a deuterium content below 90 ppm, based on the total hydrogen content, by water electrolysis using electrical power that is generated at least in part from non-fossil, renewable resources; (b) providing carbon dioxide; (c) reacting hydrogen and carbon dioxide in the presence of a catalyst to form methanol.

The present invention relates to a process for making methanol having a deuterium content below 90 ppm, based on the total hydrogen content, the methanol obtained thereby as well as to its use.

In the chemical industry methanol serves as a raw material in the production of olefines, formaldehyde, acetaldehyde, acetic acid, methyl acetate, acetic anhydride and vinyl acetate. The conventional production method involves a catalytic process using fossil feedstock such as natural gas or coal.

Synthesis gas (syngas) for the production of methanol can be produced from many sources, including natural gas, coal, biomass, or virtually any hydrocarbon feedstock, by reaction with steam (steam reforming), carbon dioxide (dry reforming) or oxygen (partial oxidation).

Syngas is for example produced from solid feedstocks via coal gasification. Coal is reacted thereby in a mixture of partial oxidation with air or pure oxygen and gasification with water vapor to give a mixture of carbon monoxide and hydrogen. Via the Boudouard equilibrium carbon monoxide is in equilibrium with carbon and carbon dioxide.

2C+O₂→2CO,

C+H₂O→CO+H₂,

C+CO₂ [symbol] 2CO,

Furthermore, the water gas shift reaction must be taken into account.

CO+H₂O [symbol] CO₂+H₂,

The exothermic reaction with oxygen provides the necessary energy to achieve the high reaction temperatures for the endothermic gasification reaction of carbon with water vapor.

In principle, beside coal, other solid feedstocks (wood, straw) can be used instead.

The most important gaseous educt for producing syngas is natural gas, which is reacted with water vapor via steam reforming. Natural gas provides the highest hydrogen to carbon monoxide ratio.

CH₄+H₂O→CO+3H₂,

Also liquid educts, such as light naphtha cuts, can be reacted, after sulfur removal, with water vapor via steam reforming.

To produce methanol, the ratio of carbon monoxide to hydrogen in the synthesis gas is adjusted to meet the reaction equation

CO+2H₂→CH₃OH,

Synthesis gas is mainly produced via steam reforming or partial oxidation of natural gas or via coal gasification. While natural gas is used for the methanol production in North America and in Europe, syngas production is based mainly on coal in China and South Africa. Depending on the carbon monoxide to hydrogen ratio, the product gases are named water gas (CO+H₂), synthesis gas (CO+2H₂) or spaltgas (CO+3H₂). Spaltgas can be hydrogen depleted or carbon monoxide enriched, for example via the water gas shift reaction by adding carbon dioxide and removing water, and water gas can be hydrogen enriched or carbon monoxide depleted in order to obtain synthesis gas.

The synthesis of methanol from CO₂ is less exothermic than that starting from synthesis gas, and it also involves as secondary reaction the reverse water-gas-shift (RWGS). To facilitate methanol synthesis, the CO in syngas is converted to CO₂ through the water-gas shift (WGS) reaction

CO₂+3H₂→CH₃OH+H₂O ΔH298K=−49.5 kJ mol−1

CO₂+H₂→CO+H₂O ΔH298K=41.2 kJ mol−1

The water-gas equilibrium mentioned above provides the basis to produce CO₂-neutral methanol if the CO₂ comes from appropriate direct or indirect biogenic sources. According to the reverse water-gas-shift (RWGS) reaction, there is the opportunity of including biogenic CO₂ directly to an adapted syngas-methanol-process. Syngas is then converted to methanol e.g. in the ranges of temperature of 250-300° C. and pressure of 5-10 MPa, using CuO/ZnO/Al₂O₃ catalyst.

In that sense CO₂ form different biogenic carbon sources could be included into the syngas to form methanol. The biogenic source of CO₂ could be from fermentation processes of biomaterial, combustion processes of biomass or waste of biobased materials or form extractive processes of atmospheric CO₂, for example by extractive regenerative process steps such as aminic CO₂ scrubbing.

Of course, mixtures of CO₂ from biogenic and fossil carbon source could be mixed to be used to produce methanol, too.

The natural isotopic abundance of ¹²C is about 98.9%, the natural isotopic abundance of ¹³C is about 1.1%. The ¹³C/¹²C isotopic ratio of chemical compounds is given relative to an international standard, the Vienna-Pee-Dee-Belemnite-Standard (V-PDB). The ¹³C/¹²C isotopic ratio is given as δ¹³C value in the unit ‰. The standard per definition has a δ¹³C value of 0‰. Substances having a higher ¹³C content than the standard have positive, substances having a lower ¹³C content than the standard have negative %6 values.

In physical organic chemistry, a kinetic isotope effect is the change in the reaction rate of a chemical reaction when one of the atoms in the reactants is replaced by one of its isotopes. Formally, it is the ratio of rate constants k_{L}/k_{H} for the reactions involving the light (k_{L}) and the heavy (k_{H}) isotopically substituted reactants (isotopologues). This change in reaction rate is a quantum mechanical effect that primarily results from heavier isotopologues having lower vibrational frequencies compared to their lighter counterparts. In most cases, this implies a greater energetic input needed for heavier isotopologues to reach the transition state, and consequently a slower reaction rate.

Isotopic rate changes are most pronounced when the relative mass change is greatest, since the effect is related to vibrational frequencies of the affected bonds. For instance, changing a hydrogen atom (H) to its isotope deuterium (D) represents a 100% increase in mass, whereas in replacing ¹²C with ¹³C, the mass increases by only 8 percent. The rate of a reaction involving a C—H bond is typically 6-10 times faster than the corresponding C-D bond, whereas a ¹²C reaction is only 4 percent faster than the corresponding ¹³C reaction.

A primary kinetic isotope effect may be found when a bond to the isotope atom is being formed or broken. A secondary kinetic isotope effect is observed when no bond to the isotope atom in the reactant is broken or formed. Secondary kinetic isotope effects tend to be much smaller than primary kinetic isotope effects; however, secondary deuterium isotope effects can be as large as 1.4 per deuterium atom.

It is an object of the present invention to provide an environmentally friendly process for producing methanol. It is a further object of the present invention to provide a methanol having a low deuterium content. The favorable kinetic isotope effect caused by the low deuterium content of the methanol may be cumulative, since it is also present in subsequent production steps further downstream in the value chain.

The object is solved by a process for making methanol having a deuterium content below 90 ppm, based on the total hydrogen content, comprising the steps:
(a) providing hydrogen with a deuterium content below 90 ppm by water electrolysis using electrical power that is generated at least in part from non-fossil, renewable resources;
(b) providing carbon dioxide;
(c) reacting hydrogen and carbon dioxide in the presence of a catalyst to form methanol.

Fossil based methanol from synthesis gas has in general δ¹³C values ranging from −50‰ to −25‰, depending on the fossil feedstock. Methanol based on carbon dioxide captured from ambient air has in general δ¹³C values ranging from −10‰ to −2.5‰, corresponding to the δ¹³C values of carbon dioxide captured from ambient air.

In preferred embodiments of the inventive process, the carbon dioxide provided in step (b) has a ¹³C-content corresponding to a δ¹³C value of >−20‰. In particular, the carbon dioxide provided in step (b) has a ¹³C-content corresponding to a δ¹³C value of from −10 to −2.5‰.

So if carbon dioxide is captured from ambient air, the ¹³C-content of the methanol corresponds to a δ¹³C value of in general >−20‰, more specifically to a δ¹³C value of from −10 to −2.5‰.

The invention also relates to methanol with a deuterium content below 90 ppm, based on the total hydrogen content. Preferably, the deuterium content is from 30 to 75 ppm, based on the total hydrogen content.

The deuterium content of hydrogen and chemical compounds containing hydrogen is given herein in atom-ppm based on the total hydrogen content (total atoms of protium ¹H and deuterium ²H).

The methanol with a deuterium content below 90 ppm, preferably from 30 to 75 ppm, based on the total hydrogen content, can be used to prepare ethylene. In general, the obtained ethylene also has a low deuterium content of below 90 ppm, preferably from 30 to 75 ppm. If carbon dioxide is captured from ambient air, the ¹³C-content of the obtained ethylene also corresponds to a δ¹³C value of in general >−20‰, more specifically to a δ¹³C value of from −10 to −2.5‰.

Electrolysis of water is an environmentally friendly method for production of hydrogen because it uses renewable H₂O and produces only pure oxygen as by-product. Additionally, water electrolysis utilizes direct current (DC) from sustainable energy resources, for example solar, wind, hydropower and biomass.

It is observed that by electrolysis of water, the deuterium atom content of the hydrogen is lower than in the hydrogen generated petrochemically, for example as contained in synthesis gas, in general below 90 ppm, preferably from 30 to 75 ppm. The deuterium atom content in electrolytically produced hydrogen may be as low as 15 ppm. The deuterium is mainly present in the form of D-H rather than D₂.

One suitable water electrolysis process is alkaline water electrolysis. Hydrogen production by alkaline water electrolysis is a well established technology up to the megawatt range for a commercial level. In alkaline water electrolysis initially at the cathode side two water molecules of alkaline solution (KOH/NaOH) are reduced to one molecule of hydrogen (H₂) and two hydroxyl ions (OH⁻). The produced H₂ emanates from the cathode surface in gaseous form and the hydroxyl ions (OH⁻) migrate under the influence of the electrical field between anode and cathode through the porous diaphragm to the anode, where they are discharged to half a molecule of oxygen (O₂) and one molecule of water (H₂O). Alkaline electrolysis operates at lower temperatures such as 30-80° C. with alkaline aqueous solution (KOH/NaOH) as the electrolyte, the concentration of the electrolyte being about 20% to 30%. The diaphragm in the middle of the electrolysis cell separates the cathode and anode and also separates the produced gases from their respective electrodes, avoiding the mixing of the produced gases. However, alkaline electrolysis has negative aspects such as limited current densities (below 400 mA/cm²), low operating pressure and low energy efficiency.

In one preferred embodiment of the inventive process, hydrogen is provided by polymer electrolyte membrane water electrolysis. Variants of polymer electrolyte membrane water electrolysis are proton exchange membrane water electrolysis (PEMWE) and anion exchange membrane water electrolysis (AEMWE).

PEM water electrolysis was developed to overcome the drawbacks of alkaline water electrolysis. PEM water electrolysis technology is similar to the PEM fuel cell technology, where solid polysulfonated membranes (Nafion®, Fumapem®) are used as an electrolyte (proton conductor). These proton exchange membranes have many advantages such as low gas permeability, high proton conductivity (0.1±0.02 S cm⁻¹), low thickness (20-300 μm), and allow high-pressure operation. In terms of sustainability and environmental impact, PEM water electrolysis is one of the most favorable methods for conversion of renewable energy to highly pure hydrogen. PEM water electrolysis has great advantages such as compact design, high current density (above 2 A cm⁻²), high efficiency, fast response, operation at low temperatures (20-80° C.) and production of ultrapure hydrogen. The state-of-the-art electrocatalysts for PEM water electrolysis are highly active noble metals such as Pt/Pd for the hydrogen evolution reaction (HER) at the cathode and IrO₂/RuO₂ for the oxygen evolution reaction (OER) at the anode.

One of the largest advantages of PEM water electrolysis is its ability to operate at high current densities. This can result in reduced operational costs, especially for systems coupled with very dynamic energy sources such as wind and solar power, where sudden spikes in energy output would otherwise result in uncaptured energy. The polymer electrolyte allows the PEM water electrolyzer to operate with a very thin membrane (ca. 100-200 μm) while still allowing high operation pressure, resulting in low ohmic losses, primarily caused by the conduction of protons across the membrane (0.1 S/cm), and a compressed hydrogen output.

The PEM water electrolyzer utilizes a solid polymer electrolyte (SPE) to conduct protons from the anode to the cathode while insulating the electrodes electrically. Under standard conditions the enthalpy required for the formation of water is 285.9 kJ/mol. One portion of the required energy for a sustained electrolysis reaction is supplied by thermal energy and the remainder is supplied through electrical energy.

The half reaction taking place on the anode side of a PEM water electrolyzer is commonly referred to as the Oxygen Evolution Reaction (OER). Here the liquid water reactant is supplied to a catalyst where it is oxidized to oxygen, protons and electrons.

The half reaction taking place on the cathode side of a PEM water electrolyzer is commonly referred to as the Hydrogen Evolution Reaction (HER). Here the protons that have moved through the membrane are reduced to gaseous hydrogen.

PEMs can be made from either pure polymer membranes or from composite membranes, where other materials are embedded in a polymer matrix. One of the most common and commercially available PEM materials is the fluoropolymer PFSA, or Nafion®, a DuPont product. While Nafion® is an ionomer with a perfluorinated backbone like Teflon, there are many other structural motifs used to make ionomers for proton-exchange membranes. Many use polyaromatic polymers, while others use partially fluorinated polymers.

An overview over hydrogen production by PEM water electrolysis is given in S. Kumar and V. Himabindu, Material Science for Energy Technologies 2 (2019), pp. 4442-4454.

An overview over hydrogen production by anion exchange membrane water electrolysis is given in H. A. Miller et al., Sustainable Energy Fuels, 2020, 4, pp. 2114-2133.

K. Harada et al., International Journal of Hydrogen Energy 45 (2020), pp. 31389-31 395 report a deuterium depletion by a factor from 2 to 3 in polymer electrolyte membrane water electrolysis. The separation factor β.

$$β = {( [ H ] / [ D ] )}_{gas} / {( [ H ] / [ D ] )}_{liquid}$$

where “gas” is the evolved gas and “liquid” is water before the electrolysis was found to be between 2 and 3 at current densities of from 1.0 to 2.0 A cm⁻², corresponding to a stoichiometric number λ of between 4 and 9 at the given water mass flow in the anode. The stoichiometric number λ is defined as follows:

$$λ = V \times ρ / ( J / 2 F \times 60 \times {M}_{H 2 O} )$$

where V (mL min⁻¹) is the water mass flow in the anode, F is the Faraday constant, J is electrolysis current (A), ρ is the density of water (g mL⁻¹) and M_{H2O} (g mol⁻¹) is the molar weight of water. A stoichiometric number λ of 10 means that 10 times the amount of fresh water than can be theoretically consumed by electrolysis at the given electrolysis current is supplied to the anode.

H. Sato et al., International Journal of Hydrogen Energy 46 (2021), pp. 33 689-33 695, report for anion exchange membrane water electrolysis that deuterium concentration in the evolving hydrogen gas is diluted by approximately 1/5 against the feed water, at λ=4.

Hence, deuterium in the evolving hydrogen gas can easily be depleted by a factor of from 2 to 5 with regard to feed water in polymer electrolyte membrane water electrolysis. Depending on the electrolysis conditions (water flow, current density), even higher depletion factors are possible. Since the average deuterium content of water is about 150 ppm, based on the total hydrogen content, hydrogen provided in step (a) of the inventive process may have a deuterium content of from 30 to 75 ppm, based on the total hydrogen content, or even lower.

The electrical power is generated at least in part from non-fossil, renewable resources. In other words, part of the electrical power can still be produced from fossil fuels, preferably from natural gas, since combustion of natural gas causes much lower carbon dioxide emission per Mega-joule of electrical energy produced than combustion of coal. However, the portion of electrical energy produced from fossil fuels should be as low as possible, preferably ≤50%, preferably ≤30%, most preferably ≤20%.

The electrical power from non-fossil resources used in water electrolysis according to the invention can be generated by nuclear energy. Nuclear energy is considered renewable by the European Commission, as long as certain preconditions (i. a. safe long-term storage of nuclear waste) are fulfilled.

The electrical power from non-fossil resources used in water electrolysis according to the invention is preferably generated from wind power, solar energy, biomass, hydropower and geothermal energy.

In one preferred embodiment of the inventive process, the electrical power used in water electrolysis is generated from hydropower. There are many forms of hydropower. Traditionally, hydroelectric power comes from constructing large hydroelectric dams and reservoirs. Small hydro systems are hydroelectric power installations that typically produce up to 50 MW of power. They are often used on small rivers or as a low-impact development on larger rivers. Run-of-the-river hydroelectricity plants derive energy from rivers without the creation of a large reservoir. The water is typically conveyed along the side of the river valley (using channels, pipes and/or tunnels) until it is high above the valley floor, whereupon it can be allowed to fall through a penstock to drive a turbine.

Wave power, which captures the energy of ocean surface waves, and tidal power, converting the energy of tides, are two forms of hydropower with future potential.

In one further preferred embodiment of the inventive process, the electrical power used in water electrolysis is generated from wind power. Wind power can be used to run wind turbines. Modern utility-scale wind turbines range from around 600 kW to 9 MW of rated power. The power available from the wind is a function of the cube of the wind speed, so as wind speed increases, power output increases up to the maximum output for the particular turbine. Areas where winds are stronger and more constant, such as offshore and high-altitude sites, are preferred locations for wind farms.

In one further preferred embodiment of the inventive process, the electrical power used in water electrolysis is generated from solar power, particularly preferred from photovoltaic systems. A photovoltaic system converts light into electrical direct current (DC) by taking advantage of the photoelectric effect. Concentrated solar power (CSP) systems use lenses or mirrors and tracking systems to focus a large area of sunlight into a small beam. CSP-Stirling has by far the highest efficiency among all solar energy technologies.

In one further preferred embodiment of the inventive process, the electrical power used in water electrolysis is generated from biomass. Biomass is biological material derived from living, or recently living organisms. It most often refers to plants or plant-derived materials which are specifically called lignocellulosic biomass. As an energy source, biomass can either be used directly via combustion to produce heat or electricity, or indirectly after converting it to various forms of biofuel. Conversion of biomass to biofuel can be achieved by different methods which are broadly classified into: thermal, chemical, and biochemical methods. Wood was the largest biomass energy source as of 2012; examples include forest residues—such as dead trees, branches and tree stumps —, yard clippings, wood chips and even municipal solid waste. Industrial biomass can be grown from numerous types of plants, including miscanthus, switchgrass, hemp, corn, poplar, willow, sorghum, sugarcane, bamboo, and a variety of tree species, ranging from eucalyptus to oil palm (palm oil).

Plant energy is produced by crops specifically grown for use as fuel that offer high biomass output per hectare with low input energy. The grain can be used for liquid transportation fuels while the straw can be burned to produce heat or electricity. Biomass can be converted to other usable forms of energy such as methane gas or transportation fuels such as ethanol and biodiesel. Rotting garbage, and agricultural and human waste, all release methane gas—also called landfill gas or biogas. Crops, such as corn and sugarcane, can be fermented to produce the transportation fuel, ethanol. Biodiesel, another transportation fuel, can be produced from left-over food products such as vegetable oils and animal fats.

In step (b) of the inventive process, carbon dioxide is provided. In preferred embodiments, the carbon dioxide that is provided in step (b) is captured from industrial flue gases or from ambient air. All available capture technologies may be used.

Capturing CO₂ is most cost-effective at point sources, such as large carbon-based energy facilities, industries with major CO₂ emissions (e.g. cement production, steelmaking), natural gas processing, synthetic fuel plants and fossil fuel-based hydrogen production plants. Extracting CO₂ from air is possible, although the lower concentration of CO₂ in air compared to combustion sources complicates the engineering and makes the process therefore more expensive.

In some preferred embodiments, the carbon dioxide that is provided in step (b) is captured from industrial flue gases.

In post combustion capture, the CO₂ is removed after combustion of the fossil fuel—this is the scheme that would apply to fossil-fuel power plants. CO₂ is captured from flue gases at power stations or other point sources. Absorption, or carbon scrubbing with amines is the dominant capture technology. It is the only carbon capture technology so far that has been used industrially.

CO₂ adsorbs to a MOF (Metal-organic framework) through physisorption or chemisorption based on the porosity and selectivity of the MOF leaving behind a CO₂ poor gas stream. The CO₂ is then stripped off the MOF using temperature swing adsorption (TSA) or pressure swing adsorption (PSA) so the MOF can be reused.

In some other preferred embodiments, the carbon dioxide that is provided in step (b) is captured from ambient air.

Direct air capture (DAC) is a process of capturing carbon dioxide (CO₂) directly from the ambient air and generating a concentrated stream of CO₂ for sequestration or utilization or production of carbon-neutral fuel. Carbon dioxide removal is achieved when ambient air makes contact with chemical media, typically an aqueous alkaline solvent or sorbents. These chemical media are subsequently stripped of CO₂ through the application of energy (namely heat), resulting in a CO₂ stream that can undergo dehydration and compression, while simultaneously regenerating the chemical media for reuse.

Dilute CO₂ can be efficiently separated using an anionic exchange polymer resin called Marathon MSA, which absorbs air CO₂ when dry, and releases it when exposed to moisture. A large part of the energy for the process is supplied by the latent heat of phase change of water. Other substances which can be used are metal-organic frameworks (or MOFs). Membrane separation of CO₂ rely on semi-permeable membranes.

In step (c), hydrogen and carbon dioxide are reacted in the presence of a catalyst to form methanol.

An overview of suitable catalyst systems is given by Kristian Stangeland, Hailong Li & Zhixin Yu, Energy, Ecology and Environment volume 5, pages 272-285 (2020). Multi-component catalyst systems are required for this process. The interaction between components is essential for high activity and selectivity of CO₂-to-methanol catalysts. This has been demonstrated by numerous catalyst systems comprised of various metals (i.e., Cu, Pd, Ni) and metal oxides (i.e., ZnO, ZrO₂, In₂O₃). These complex systems can contain a mixture of metallic, alloy, and metal oxide phases. The most promising catalyst systems for large-scale industrial processes are currently Cu-based and In-based catalysts due to their superior catalytic performance.

A process for the CO₂-to-methanol synthesis can be carried out, for example, by the method known from DE-A-42 20 865, which produces methanol under the influence of silent electrical discharges.

Alternatively, methanol synthesis can also be carried out in a thermal reactor under pressure and elevated temperature and in the presence of a copper-based catalyst (DE 43 32 789 A1; DE 19739773 A1).

Typical catalysts are described, for example, in the publication by N. Kanoun et al. “Catalytic properties of Cu based catalysts containing Zr and/or V for methanol synthesis from a carbon dioxide and hydrogen mixture” in CATALYSIS LETTERS 15, (1992) 231-235. Potential catalysts like CuO/ZnO and Cu—ZnO—Al₂O₃ are also described by R. M. Navarro et al. “Methanol Synthesis from CO₂: A Review of the Latest Developments in Heterogeneous Catalysis” Materials (2019), 12, 3902 and in “Catalytic carbon dioxide hydrogenation to methanol: A review of recent studies” in chemical engineering research and design 92 (2014) 2557-2567.

Recently a high selective catalysts In₂O₃/ZrO₂ was described for industrial relevant conditions. A typical range of industrially relevant conditions for the hydrogenation of CO₂ to methanol are T=200-300° C., P=10-50 MPa, and gas hourly space velocity (GHSV) of 16 000-48000 h⁻¹ (Angew. Chem. Int. Ed. 2016, 55, 6261-6265).

Step (c) can be carried out in the presence of a copper-zinc-alumina catalyst. If copper-zinc-alumina catalysts are employed, the preferred temperature is in the range of from 150 to 300° C., preferably 175 to 300° C. and the preferred pressure is in the range of from 10 to 150 bar (abs).

In general, ethylene is produced from methanol in a methanol to olefin-process (MTO-process). Since the process involves the cleavage of C—H bonds and C-D bonds, respectively, the related primary isotope effect will be pronounced. In the MTO process, a mixture of ethylene and propylene is produced from methanol on a highly selective silicon alumina phosphate zeolith-catalyst in fluid bed operation. The ratio propylene to ethylene can be adjusted by chosing appropriate process conditions, and may vary from 0.77 in the ethylene production mode and 1.33 in the propylene production mode.

The overall kinetic isotope effect is cumulative, since it will also be present in all subsequent production steps downstream the value chain.

The deuterium content of the hydrogen produced by electrolysis and the deuterium content of the methanol obtained according to the invention can be determined via mass spectrometry. If hydrogen is produced by electrolysis according to the invention, its deuterium content is below 90 ppm. Thus, it is possible to determine by the deuterium content of methanol whether the methanol was produced with hydrogen from water-electrolysis or conventionally, e.g. via synthesis gas from natural gas.

The methanol with a deuterium content below 90 ppm, based on the total hydrogen content, obtainable according to the invention, can be used for fuel applications, such as the production of MTBE/TAME, gasoline blending, the production of dimethyl ether and the production of biodiesel, for producing formaldehyde, acetic acid, olefins, sodium methylate, methylation products, such as dimethylphenol, for the production of dimethylterephthalat and methyl mercaptane.

The formaldehyde obtained from the methanol of the invention can be further used e. g. for the production of polyoxymethylene, butinediol, methylendiphenyldiisocyanate, neopentylglycol, methacrylic acid, phenol formaldehyde resins, urea condensation resins, melamine resins and acetone resins.

The acetic acid obtained from the methanol of the invention can be further used e. g. for the production of chloroacetic acid, acetic acid esters, methylisopropylketone and acetic acid anhydride.

The methyl mercaptane obtained from the methanol of the invention can be further used e. g. for the production of methylmercaptopropionaldehyde, dimethyldisulfide and methanesulfonic acid.

In particular, the present invention also relates to the use of methanol with a deuterium content below 90 ppm, based on the total hydrogen content, obtainable according to the process of the invention, to produce formaldehyde, acetic acid, methylamine, methyl-tert.-butylether, methyl methacrylate, trimethylolpropane, methyl chloride, methylchkorosilanes and silicones.

The present invention also relates to a process for producing formaldehyde comprising steps (a) to (c) as described above and the additional step
(d) dehydrogenation or oxidation of the methanol obtained in step (c) to give formaldehyde.

Formaldehyde is produced industrially by the catalytic oxidation of methanol. The most common catalysts are silver metal, iron(III) oxide, iron molybdenum oxides or vanadium oxides. In the commonly used formox process, methanol and oxygen react at ca. 250-400° C. in presence of iron oxide in combination with molybdenum and/or vanadium to produce formaldehyde according to the chemical equation:

$$2 {CH}_{3} OH + {O}_{2} \to 2 {CH}_{2} O + 2 {H}_{2} O$$

The silver-based catalyst usually operates at a higher temperature, about 650° C. Two chemical reactions on it simultaneously produce formaldehyde: that shown above and the dehydrogenation reaction:

$${CH}_{3} OH \to {CH}_{2} O + {H}_{2}$$

The formaldehyde obtained in step (d) can be used to produce trimethylolpropane. In a further aspect, the invention also relates to a process for producing trimethylolpropane comprising steps (a) to (d) and the additional step (e) reacting formaldehyde obtained in step (d) with butanal to give trimethylolpropane.

Trimethyolpropane is produced via a two step process, starting with the condensation of butanal with formaldehyde:

CH₃CH₂CH₂CHO+2CH₂O→CH₃CH₂C(CH₂OH)₂CHO

The second step entails a Cannizzaro reaction:

CH₃CH₂C(CH₂OH)₂CHO+CH₂O+NaOH→CH₃CH₂C(CH₂OH)₃+NaO₂CH

The present invention also relates to a process for producing acetic acid comprising steps (a) to (c) as describe above and the additional step
(d) reacting methanol obtained in step (c) with carbon monoxide to give acetic acid.

Acetic acid can be produced by carbonylation of methanol in step (d). The process involves iodomethane as an intermediate, and occurs in three steps. A catalyst, metal carbonyl, is needed for the carbonylation (step 2).
1. CH₃OH+HI→CH₃I+H₂O
2. CH₃I+CO→CH₃COI
3. CH₃COI+H₂O→CH₃COOH+HI

Two related processes exist for the carbonylation of methanol: the rhodium-catalyzed Monsanto process, and the iridium-catalyzed Cativa process.

The Monsanto process operates at a pressure of 30-60 atm and a temperature of 150-200° C. and gives a selectivity greater than 99%. The catalytically active species is the anion cis-[Rh(CO)₂I₂]⁻. The first organometallic step is the oxidative addition of methyl iodide to cis-[Rh(CO)₂I₂]⁻ to form the hexacoordinate species [(CH₃)Rh(CO)₂I₂]⁻. This anion rapidly transforms, via the migration of a methyl group to an adjacent carbonyl ligand, affording the pentacoordinate acetyl complex [(CH₃CO)Rh(CO)I₃]⁻. This five-coordinate complex then reacts with carbon monoxide to form the six-coordinate dicarbonyl complex, which undergoes reductive elimination to release acetyl iodide (CH₃C(O)I). The catalytic cycle involves two non-organometallic steps: conversion of methanol to methyl iodide and the hydrolysis of the acetyl iodide to acetic acid and hydrogen iodide.

The Cativa process is a further method for the production of acetic acid by the carbonylation of methanol. The technology is similar to the Monsanto process. The process is based on an iridium-containing catalyst, such as the complex [Ir(CO)₂I₂]⁻. The catalytic cycle for the Cativa process begins with the reaction of methyl iodide with the square planar active catalyst species to form the octahedral iridium(III) species [Ir(CO)₂(CH₃)I₃]⁻. This oxidative addition reaction involves the formal insertion of the iridium(I) centre into the carbon-iodine bond of methyl iodide. After ligand exchange of iodide for carbon monoxide, the migratory insertion of carbon monoxide into the iridium-carbon bond results in the formation of a species with a bound acetyl ligand. The active catalyst species is regenerated by the reductive elimination of acetyl iodide. The acetyl iodide is hydrolysed to produce the acetic acid product, in the process generating hydroiodic acid which is in turn used to convert the starting material methanol to the methyl iodide used in the first step.

The present invention also relates to a process for producing methylamine comprising steps (a) to (c) as described above and the additional step
(d) reacting methanol obtained in step (c) with ammonia to give methylamine.

Step (d) can be carried out at 350 to 450° C. and 15 to 25 bar in the presence of a catalyst containing Al₂O₃ and SiO₂.

The present invention also relates to a process for producing methyl-tert.-butylether comprising steps (a) to (c) as described above and the additional step
(d) reacting methanol obtained in step (c) with isobutene to give methyl-tert.-butylether.

Step (d) can be carried out at 40 to 90° C. and 3 to 20 bar in the presence of an acidic ion exchanger.

The present invention also relates to a process for producing methyl methacrylate comprising steps (a) to (c) as described above and the additional step
(d) reacting methanol obtained in step (c) with methacrylic acid to give methyl methacrylate.

1.-22. (canceled)

23. A process of making methanol having a deuterium content below 90 ppm, based on the total hydrogen content, comprising the steps:
(a) providing hydrogen with a deuterium content below 90 ppm, based on the total hydrogen content, by water electrolysis using electrical power that is generated at least in part from non-fossil, renewable resources;
(b) providing carbon dioxide; and
(c) reacting hydrogen and carbon dioxide in the presence of a catalyst to form methanol.

24. The process of claim 23, wherein the electrical power is generated from at least one of wind power, solar energy, biomass, hydropower and geothermal energy.

25. The process according to claim 23, wherein hydrogen is provided by polymer electrolyte membrane water electrolysis.

26. The process according to claim 25, wherein hydrogen is provided by one of proton exchange membrane water electrolysis (PEMWE) and anion exchange membrane water electrolysis (AEMWE).

27. The process according to claim 23, wherein step (b) further comprises capturing the carbon dioxide from industrial flue gases

28. The process according to claim 23, wherein step (b) further comprises capturing the carbon dioxide from ambient air.

29. The process according to claim 23, wherein step (c) is carried out in the presence of a copper-zinc-alumina catalyst.

30. The process according to claim 23, wherein the hydrogen provided in step (a) has a deuterium content of from 30 to 75 ppm.

31. The process according to claim 23, wherein the carbon dioxide provided in step (b) has a ¹³C-content corresponding to a δ¹³C value of −10 to −2.5‰.

32. The process according to claim 23, wherein the methanol has a deuterium content below 90 ppm, based on the total hydrogen content.

33. The process according to claim 32, wherein the methanol has a deuterium content of 30 to 75 ppm, based on the total hydrogen content.

34. The process of claim 32, wherein the methanol has a ¹³C-content corresponding to a δ¹³C value of −10 to −2.5‰.

35. A process for producing formaldehyde comprising steps (a) to (c) as defined in claim 23 and the additional step:
(d) dehydrogenation or oxidation of the methanol obtained in step (c) to give formaldehyde.

36. A process for producing trimethylolpropane comprising steps (a) to (d) as defined in claim 37 and the additional step:
(e) reacting formaldehyde obtained in step (d) with butanal to give trimethylolpropane.

37. A process for producing acetic acid comprising steps (a) to (c) as defined in claim 23 and the additional step:
(d) reacting methanol obtained in step (c) with carbon monoxide to give acetic acid.

38. A process for producing methylamine comprising steps (a) to (c) as defined in claim 23 and the additional step:
(d) reacting methanol obtained in step (c) with ammonia to give methylamine.

39. The process according to claim 38, wherein step (d) is carried out at 350 to 450° C. and 15 to 25 bar in the presence of a catalyst containing Al₂O₃ and SiO₂.

40. A process for producing methyl-tert.-butylether comprising steps (a) to (c) as defined in claim 23 and the additional step:
(d) reacting methanol obtained in step (c) with isobutene to give methyl-tert.-butylether.

41. The process according to claim 40, wherein step (d) is carried out at 40 to 90° C. and 3 to 20 bar in the presence of an acidic ion exchanger.

42. A process for producing methyl methacrylate comprising steps (a) to (c) as defined in claim 23 and the additional step:
(d) reacting methanol obtained in step (c) with methacrylic acid to give methyl methacrylate.
